(19) United States
(12) Patent Application Publication
(10) Pub. No.: US 2020/0171116 A1
(43) Pub. Date: Jun. 4, 2020

(54) PSOHEMPSIS

(71) Applicant: Simeon Augustus Marcellin, Brooklyn, NY (US)

(72) Inventor: Simeon Augustus Marcellin, Brooklyn, NY (US)

(21) Appl. No.: 16/748,380

(22) Filed: Jan. 21, 2020

Related U.S. Application Data
(63) Continuation of application No. 62/892,957, filed on Aug. 28, 2019.

(51) Int. Cl. A61K 36/73; A61K 9/06; A61K 47/14; A61K 9/00

(52) U.S. Cl. CPC A61K 36/73; A61K 9/06; A61K 2236/331; A61K 9/0014; A61K 47/14

(57) ABSTRACT

1)The name of My Invention is PSOHEMPSIS, It's Intended used and propose are Individuals suffering from PSORIASIS

2) PSOHEMPSIS Invention is used for People with PSORIASIS on Surface of Skin.

3) The Description of how PSOHEMPSIS works, apply Topically on Skin of Psoriasis twice a day.

4) PSOHEMPSIS (SHOULD NOT BE USE OR APPLY IN ANY OTHER WAY AS DESCRIBED).

### BACKGROUND OF INVENTION

To whom it may Concern,

Background of Invention for more than 30 years I have been helping people with DISEASE'S in fact mentoring Individuals back to health. My Education, I earn a Bachelor degree in Science, additionally I earn a Master's degree in Theology. I consider myself gifted in health wellness. In the past I have use the Medicine INDIAN HEMP HERB MEDICINE apply Topical on surface of skin to Individual with PSORIASIS. The (Advantages) of topical use of Medicine PSOHEMPSIS on surface of the skin PATHOLOGY RASH, ERUPTION REDNESS on the skin have Disappear or totally heal in 3 days. (Existing problems) individuals with Psoriasis use other products for up to 3 months but Rash, Eruptions, and Redness still accrued. There are no existing (problems) with PSOHEMPSIS. I truly believe my Invention PSOHEMPSIS for PSORIASIS can help alleviate countless suffering.

### DETAIL DESCRIPTION COMPOSITION OF INVENTION PSOHEMPSIS

The Invention process for PSOHEMPSIS; Boil INDIAN HEMP HERB to 212* Fahrenheit for 5 minutes (212*f−32)×5/9=100* Celsius. Aloud five hours too cool.

1. (PSOHEMPSIS Liquid form) Add POTASSIUM SORBATE as a PRESERVATIVE. (Fragrance Optional)

### 2. (PSOHEMPSIS Cream) Add COCOA BUTTER and or VEGETABLE OIL and POTASSIUM SORBATE as a PRESERVATIVE. (Fragrance Optional)

1. CLAIMS SET FORTH, PSOHEMPSIS the Invention LIQUID FORM Apply TOPICAL on SKIN with Psoriasis will go in Remission Disappear in (3) days.
PSOHEMPSIS the Invention Cream Apply Externally to Surface of Skin with PSORIASIS will go in Remission Disappear in (3) days.
PSOHEMPSIS the Invention Cream Apply TOPICAL on SKIN with PSORIASIS will go in Remission Disappear in (3) days
